(19) United States
(12) Patent Application Publication
(10) Pub. No.: US 2025/0129303 A1
(43) Pub. Date: Apr. 24, 2025

(54) Process and Plant for Producing One or More Hydrocarbons

(71) Applicant: Linde GmbH, Pullach (DE)

(72) Inventors: Gunther Kracker, Weyarn (DE); Mathieu Zellhuber, Planegg (DE); Sean McCracken, Puchheim (DE); Andreas Meiswinkel, Rimsting (DE); Martin Schubert, München (DE); Desislava Tota, München (DE)

(21) Appl. No.: 18/717,908

(22) PCT Filed: Dec. 8, 2022
(86) PCT No.: PCT/EP2022/084977

§ 371 Date: Jan. 9, 2025

(30) Foreign Application Priority Data
Dec. 8, 2021 (EP) 21020623.1

(51) Int. Cl. C10G 55/06; B01J 23/28; C10G 70/02

(52) U.S. Cl. CPC C10G 55/06; B01J 23/28; C10G 70/02; B01J 2523/55; B01J 2523/56; B01J 2523/68; C10G 2300/104

(57) ABSTRACT

A method for producing hydrocarbons includes subjecting a first feed stream to steam cracking to obtain a first product stream, and subjecting an ethane-containing second feed stream to oxidative dehydrogenation to obtain a second product stream. A portion of the first product stream is subjected to a deethanization or a depropanization separately from the second product to obtain a lighter fraction and a heavier fraction. A demethanization feed stream is formed by combining at least a portion of the lighter fraction and at least a portion of the second product stream and is subjected at least in part to demethanization. Partial oxygen removal is carried out during the formation of the demethanization feed stream. The oxygen removal is carried out downstream of the combining step.

### CROSS-REFERENCE TO RELATED APPLICATIONS

This application is the national phase of, and claims priority to, International Application No. PCT/EP2022/084977, filed 8 Dec. 2022, which claims priority to European Patent Application No. EP21020623.1, filed 8 Dec. 2021.

### FIELD OF THE INVENTION

The invention relates to a method and a system for producing one or more hydrocarbons.

### BACKGROUND

Steam cracking of hydrocarbons is described, for example, in the article “Ethylene” in Ullmann's Encyclopedia of Industrial Chemistry, online edition, Apr. 15, 2009, DOI: 10.1002/14356007.a10_045.pub2. Steam cracking is used primarily for obtaining short-chain olefins, such as ethylene and propylene, diolefins, such as butadiene, or aromatics, but is not limited thereto.

The oxidative dehydrogenation (ODH) of paraffins with two to four carbon atoms (in the case of the oxidative dehydrogenation of ethane to ethylene also called ODHE or ODH-E) is also known. During oxidative dehydrogenation, said paraffins are converted with oxygen, inter alia, to give the respective olefins and water. It represents an alternative and in certain cases advantageous method for the production of olefins.

As explained below, combination methods of steam cracking and oxidative dehydrogenation are also known, for example from WO 2018/024650 A1, WO 2014/134703 A1 and CN 103086824 B.

### SUMMARY

An object of the invention is to improve corresponding combination methods.

According to an embodiment, a method for producing one or more hydrocarbons includes subjecting a first feed stream to steam cracking to obtain a first product stream; subjecting a second feed stream containing ethane to oxidative dehydrogenation to obtain a second product stream; subjecting at least a portion of the first product stream to deethanization or depropanization separately from the second product stream to obtain a lighter fraction and a heavier fraction; forming a demethanization feed stream by combining at least a portion of the lighter fraction and at least a portion of the second product stream; and subjecting the demethanization feed stream to demethanization at least in part. At least partial oxygen removal is carried out during the formation of the demethanization feed stream, the oxygen removal being carried out downstream of the combining step. Further, at least a portion of the first product stream is fed to the combining step without prior acetylene hydrogenation or with only partial acetylene hydrogenation. The oxygen removal comprises an acetylene removal.

### BRIEF DESCRIPTION OF THE DRAWINGS

FIG. 1 illustrates a method according to a comparative example not according to the invention.

FIG. 2 illustrates a method according to a comparative example not according to the invention.

FIG. 3 illustrates a method according to a comparative example not according to the invention.

FIG. 4 illustrates a method according to a comparative example not according to the invention.

FIG. 5 illustrates a method according to an embodiment of the invention.

FIG. 6 illustrates a method according to an embodiment of the invention.

FIG. 7 illustrates a method according to a further embodiment.

### WRITTEN DESCRIPTION

A method for producing one or more hydrocarbons is proposed, in which a first feed stream is subjected to steam cracking to obtain a first product stream, and a second feed stream containing ethane is subjected to oxidative dehydrogenation to obtain a second product stream, a demethanization feed stream being formed using at least a portion of the first product stream and using at least a portion of the second product stream, which demethanization feed stream is subjected to demethanization at least in part, and in which oxygen removal is carried out at least in part during the formation of the demethanization feed stream. At least a portion of the first product stream is subjected to deethanization or depropanization to obtain a lighter fraction and a heavier fraction separately from the second product stream, the demethanization feed stream being formed by combining at least a portion of the lighter fraction and at least a portion of the second product stream, and the oxygen removal being carried out downstream of the step of combining.

It is proposed that at least a portion of the first product stream is fed to the combining step without prior acetylene hydrogenation or with only partial acetylene hydrogenation, and that the oxygen removal comprises acetylene removal.

A particular advantage of such a procedure is that a separate acetylene hydrogenation in the first product stream of the steam cracker can also be omitted entirely, provided that the oxygen removal is carried out in such a way that in this case a corresponding acetylene removal in accordance with the specifications is also achieved (which is usually the case). Therefore, there is no need to provide the corresponding equipment at great expense. For further advantages, reference is made to the detailed description of the invention and its variants below.

Overall, according to embodiments of the invention, an optimized integration of an oxidative dehydrogenation of ethane and steam cracking is provided. In particular, there is the advantage of jointly using certain method steps or components, in particular demethanization and separation from one another of hydrocarbons having two carbon atoms (C2 splitter). For example, in embodiments of the invention there is also the possibility of expanding the capacity of a steam cracker by using the oxidative dehydrogenation of ethane, in particular for ethane recycling. Steam crackers designed for pure or predominantly liquid feedstocks can also be expanded to include a corresponding ethane recycling system. By oxidative dehydrogenation of ethane, acetic acid can additionally be provided in integrated complexes. In particular, it is possible to optimize and adjust the ethylene capacity according to requirements. Furthermore, a reduction in the requirement for fresh water as a feedstock, in particular for the steam cracker, is possible by recovering additional water formed during the oxidative dehydrogenation of ethane. In the oxidative dehydrogenation of ethane, significantly more water is formed than is supplied. This additional water can then be fed in part into the steam cracker's water feed stream, resulting in further environmental and cost benefits for fresh and waste water.

In a comparative example not according to the invention—hereinafter also referred to in particular as “variant 1”—it is provided that, by combining at least a portion of the first product stream and at least a portion of the second product stream without prior separation of gaseous hydrocarbons, a collective stream is formed and is subjected to carbon dioxide removal at least in part, the demethanization feed stream being formed using at least a portion of a withdrawal stream taken from the carbon dioxide removal. In this embodiment, it is possible, in particular, to jointly use the carbon dioxide removal and all components downstream of a corresponding carbon dioxide removal. However, in this first comparative example, the advantages of oxygen removal occurring downstream of the step of combining cannot be achieved.

In an alternative second comparative example not according to the invention,

and embodiments of the invention—hereinafter also referred to in particular as “variant 2” and “variant 3”—it is provided that at least a portion of the first product stream is subjected to deethanization or depropanization to obtain a lighter fraction and a heavier fraction separately from the second product stream, the demethanization feed stream being formed by combining at least a portion of the lighter fraction and at least a portion of the second product stream. This group of embodiments results in particular a simplification of the raw gas treatment of the oxidative dehydrogenation of ethane before corresponding combining, since oxygen does not have to be removed extremely deeply. However, oxygen depletion is advantageously carried out to a value that is sufficiently low to avoid the need for pressure-resistant or explosion-proof design of subsequent system components, such as in demethanization. Any remaining oxygen content in the demethanization feed stream can be removed in the demethanization. This results in a minimization of ethylene loss upstream, especially during demethanization.

In general, the term “raw gas treatment” is understood to mean a treatment of the product stream from oxidative dehydrogenation, or a stream derived therefrom, regardless of where in a corresponding process it takes place. However, raw gas treatment in the sense understood here always takes place before it is combined with a stream from steam cracking.

In the comparative examples not according to the invention—namely in particular according to the already mentioned “variant 1” and according to the already mentioned “variant 2”—it is provided in particular that the second product stream is subjected to a treatment upstream of the step of combining, which comprises the removal of oxygen. In this embodiment, the oxygen removal needs to be adapted only to the requirements in the product stream of the oxidative dehydrogenation.

In such method variants not according to the invention—that is to say in particular according to the already mentioned “variant 1” and according to the already mentioned “variant 2”—it is provided in particular that the processing comprises one or more additional steps selected from condensate separation, pre-compression, carbon dioxide removal, and drying. In this way, a typically much higher carbon dioxide content in the product stream of the oxidative dehydrogenation can be significantly reduced in advance, in order to then carry out the (further) carbon dioxide separation in the form of a fine purification after the step of combining. The condensate separation enables in particular the above-mentioned extraction of acetic acid as a valuable product and water as a feedstock.

Within the scope of the invention—namely in particular according to the already mentioned “variant 3”—it is provided that the oxygen removal is carried out downstream of the combining step. As mentioned, these measures can result in it being possible for a separate acetylene hydrogenation in the first product stream of the steam cracker to also be omitted entirely, provided that the oxygen removal is carried out in such a way that a corresponding acetylene removal in accordance with the specifications is also achieved (which is usually the case). The invention provides for this, i.e., relates to a method in which at least a portion of the first product stream is fed to the combining step without prior acetylene hydrogenation or with only partial acetylene hydrogenation, and in which the oxygen removal comprises acetylene removal.

This is in particular synonymous with the lighter fraction or the portion thereof which is subjected to the combining step having a content of, for example, 500 vol.ppm to 30,000 vol.ppm (millionths of parts by volume) of acetylene. A corresponding value can also be 2000 vol.ppm to 25,000 vol.ppm, or 3000 vol.ppm to 20,000 vol.ppm of acetylene, it also being possible for the acetylene content to reach an upper limit of 15,000 vol.ppm.

After such a combined oxygen and acetylene removal, a content of significantly less than 1 vol.ppm acetylene is particularly desirable. This is in particular the case because acetylene is later concentrated in the ethylene when lighter components and ethane are separated. The target values in the ethylene product are typically less than 2 vol.ppm and possibly even less than 1 vol.ppm. In the case of partial removal during oxygen removal (i.e., if further acetylene hydrogenation takes place later), higher values are permissible.

In any case, however, the acetylene content is reduced compared to the original fission gas.

In the case of subsequent acetylene hydrogenation as part of a corresponding hydrogenation, a mixture of substances removed from the oxygen removal process may have a content of less than 250 vol.ppm, less than 100 vol.ppm, or less than 10 vol.ppm of acetylene. If this subsequent acetylene removal does not take place, the acetylene content may in particular be less than 5 vol.ppm, less than 2 vol.ppm, less than 1 vol.ppm, less than 0.5 vol.ppm, less than 0.3 vol.ppm, or less than 0.1 vol.ppm. In both cases, oxygen removal is carried out in such a way that a corresponding reduction in the acetylene content is achieved. Residual oxygen contents may in particular be less than 1000 vol.ppm, less than 500 vol.ppm, less than 100 vol.ppm, less than 10 vol.ppm, or less than 1 vol.ppm.

In such an embodiment of the invention—i.e., in particular according to the already mentioned “variant 3”—it is provided in particular that the second product stream is subjected to condensate separation and/or pre-compression upstream of the step of combining, and/or in which one or more method steps are carried out downstream of the oxygen removal and upstream of the demethanization, which step(s) is or are selected from carbon dioxide removal, drying, and hydrocarbon fractionation. Thus, here too, in addition to a first carbon dioxide removal in the first product stream, a second carbon dioxide removal can be carried out, in order not to exceed or violate the existing capacity limits for processing the first product stream.

In one embodiment of the invention, it is provided that, in the demethanization, a fraction is formed which predominantly or exclusively contains hydrocarbons having two carbon atoms and which is subjected to separation from one another of the hydrocarbons having two carbon atoms, after or before a selective hydrogenation of acetylene. As has been recognized in the present case, selective hydrogenation downstream of a steam cracker is often capacity-limiting during retrofitting or capacity expansion. However, if this is provided substantially largely downstream of the demethanization, in particular, as is the case in embodiments of the invention, the latter is subjected to only a minor additional load.

According to one embodiment of the invention, a catalyst containing at least palladium is used in the selective hydrogenation. Particularly advantageous and technically common catalysts are based on palladium, but can additionally also be doped with other elements, in particular silver, gold, cerium, etc., in order to further improve the catalytic properties.

According to one embodiment of the invention, the oxidative dehydrogenation is carried out using one or more catalysts containing the metals molybdenum, vanadium, niobium and optionally tellurium. As explained below, corresponding catalyst systems are proven and robust, and, in addition to an advantageous extraction of olefins, also optionally allow the production of corresponding organic acids in co-production.

In principle, carbon dioxide removal, to which a collecting stream is subjected, for example, in comparative examples not according to the invention, can be carried out in the form of a caustic wash or can comprise such a wash. As also explained below, a separate carbon dioxide removal can be provided in the second product stream, in particular before a step of combination to form a corresponding collecting stream—in particular according to the already mentioned “variant 1”—so that the existing carbon dioxide removal is not excessively burdened or too much caustic solution is required.

In the comparative examples not according to the invention, it can be provided that the carbon dioxide removal to which the second product stream is subjected—in particular according to the already mentioned “variant 1” and according to the already mentioned “variant 2”—or in the embodiments of the invention the carbon dioxide removal downstream of the oxygen removal-in particular according to the already mentioned “variant 3”—is carried out in the form of regenerative scrubbing or comprises such scrubbing. This regenerative scrubbing can in particular handle higher carbon dioxide contents and is advantageous in terms of operating resources, but typically does not deplete the corresponding gas mixture of carbon dioxide to the same extent as a caustic wash. If necessary, a caustic wash can therefore be installed downstream for fine cleaning.

According to one embodiment of the invention, the oxygen removal is carried out in particular such that a residual oxygen content downstream thereof is less than 500 vol.ppm, 250 vol.ppm, 100 vol.ppm, 10 vol.ppm, or 1 vol.ppm. In particular, as mentioned, a higher oxygen content is possible here than is typically the case in the raw gas stream of a steam cracker. Nevertheless, the value is advantageously sufficiently low to avoid a safety hazard due to enrichment in a light fraction of methane, carbon monoxide and other low-boiling components, thus resulting in safety advantages. It must also be ensured that sufficient depletion is carried out to avoid fouling, especially in the presence of dienes. Because oxygen does not have to be removed extremely deeply and any remaining residual content is removed in a demethanization step, in particular the ethylene loss in the raw gas treatment is minimized.

In the separation of hydrocarbons having two carbon atoms, an ethane-enriched stream is recovered, the ethane-enriched stream being recycled at least in part to steam cracking and/or oxidative dehydrogenation as part of the first and/or second feed stream. In the case of such an embodiment, it is possible to expand the capacity of a steam cracker by using oxidative dehydrogenation, in particular for ethane recycling, as mentioned above.

According to one embodiment of the invention, in the processing downstream of the respective combining step, at least a portion of the gas mixture being processed in each case is cooled to temperatures of less than −120° C., −135° C., or −150° C. In particular, it is possible to use peak cold in demethanization, which results in a reduction of ethylene losses in the light fraction from demethanization.

According to one embodiment of the invention, at least a portion of the second product stream is subjected to the above-mentioned condensate separation, in which a condensate stream is separated which contains at least 1 wt. % of acetic acid, the condensate stream being in particular subjected to further processing in order to obtain acetic acid as a valuable product. The invention enables, in a corresponding embodiment, an advantageous extraction of this valuable product. As explained in more detail below, acetic acid can also be hydrogenated in particular to increase the ethylene yield.

According to one embodiment of the invention, a gaseous and/or liquid feed is fed to the steam cracking. In particular when used in liquid form for steam cracking, the ethane can be fed to the oxidative dehydrogenation.

According to one embodiment of the invention, hydrogenation of acetic acid formed in particular in the oxidative dehydrogenation can be provided. Alternatively or additionally, dehydration of ethanol, in particular formed in the dehydrogenation of acetic acid, may be provided. This can in particular increase the ethylene yield.

The invention also relates to a system which is designed to carry out a method in any embodiment of the invention, and said system benefits in the same way from the advantages mentioned above for individual embodiments and explained below. This is a system for producing one or more hydrocarbons, which is designed to subject a first feed stream to steam cracking to obtain a first product stream, and a second feed stream containing ethane to oxidative dehydrogenation to obtain a second product stream, to form a demethanization feed stream using at least a portion of the first product stream and using at least a portion of the second product stream, and to subject said demethenization feed stream to demethanization at least in part, and to carry out at least partial oxygen removal during the formation of the demethanization feed stream, the system being designed to subject at least a portion of the first product stream to deethanization or depropanization to obtain a lighter fraction and a heavier fraction separately from the second product stream, to form the demethanization feed stream by combining at least a portion of the lighter fraction and at least a portion of the second product stream, and to carry out the oxygen removal downstream of the step of combining. It is further designed to feed at least a portion of the first product stream to the step of combining, without prior acetylene hydrogenation or with only partial acetylene hydrogenation, and to carry out the oxygen removal as an acetylene removal.

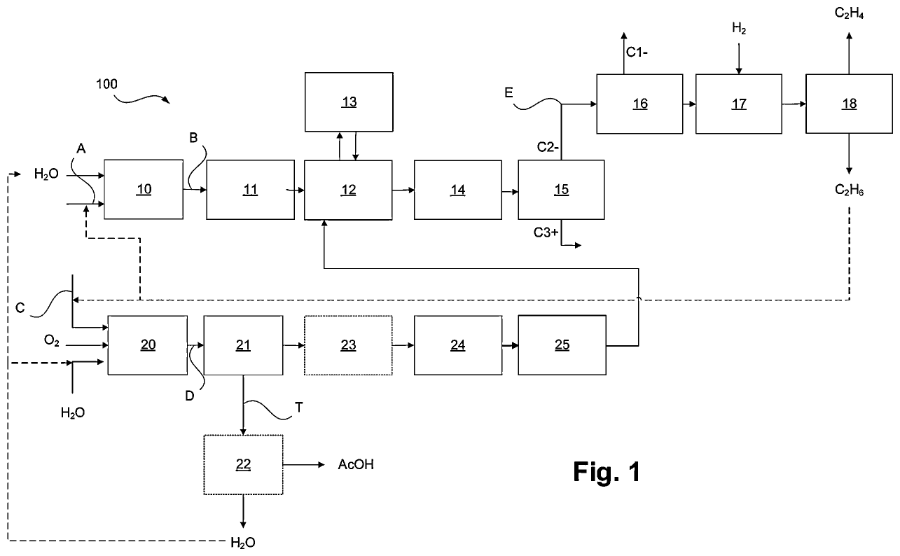

Referring now to FIG. 1, a method according to a comparative example not according to the invention is shown in the form of a schematic flowchart and is designated as a whole by 100.

The central method steps illustrated here are steam cracking 10 using one or more process units such as cracking furnaces and oxidative dehydrogenation 20, which can be carried out using one or more reactors. As illustrated here, a first feed stream, designated here by A, is fed to the steam cracker 10 and is processed there to obtain a first product stream, designated here by B. Water (H₂O) in the form of steam is also fed to the steam cracker 10. A second feed stream, designated here by C, containing ethane, is subjected to oxidative dehydrogenation 20 to obtain a second product stream, designated here by D. Water (H₂O) in the form of steam, and oxygen (O₂), are also fed to the oxidative dehydrogenation 20. The routing of other material flows, which are not separately designated, is immediately apparent from the representation in the form of flow arrows.

The steam cracking step 10 is followed by a quenching step 11, which is standard in the industry for example. Downstream of the quenching step 11 there is a compression 12, a removal 13 of carbon dioxide (usually) carried out at an intermediate stage of the compression 12, a drying 14, a deethanization 15 to obtain a heavier fraction C3+ containing hydrocarbons having three carbon atoms and heavier hydrocarbons, and a lighter fraction C2− containing hydrocarbons having two carbon atoms, methane, carbon monoxide and possibly other low-boiling components (such as remaining oxygen), a demethanization 16 to separate a light fraction C1− containing methane and other low-boiling components, a tail-end hydrogenation 17 to which hydrogen (H₂) is fed, and a separation 18 from one another of hydrocarbons having two carbon atoms to obtain an ethylene fraction (C₂H₄) and an ethane fraction (C₂H₆). The latter can be recycled to steam cracking 10 and/or oxidative dehydrogenation 20, as illustrated by a dashed arrow.

The oxidative dehydrogenation 20 is followed by a condensate separation 21 in which a condensate stream T is obtained. This is fed to an optional condensate treatment 22, in which an acetic acid fraction (AcOH) and a water fraction (H₂O) can be obtained. The latter can be recycled to steam cracking 10 and/or oxidative dehydrogenation 20, as illustrated by a dashed arrow. The separation 21 of condensate is followed here by an optional (pre-)compression 23, downstream of which an oxygen removal 24 and a removal 25 of carbon dioxide follow. A trace removal 24 can also take place before a (pre-)compression 23 or at an intermediate stage of the (pre-)compression 23.

In the embodiment 100 illustrated in FIG. 1, a gas mixture present downstream of the removal 25 of carbon dioxide is led into the compression step 12, which is arranged upstream of the demethanization 16, so that a demethanization feed stream, designated here by E, is formed using a portion each of the first product stream B and the second product stream D, and the oxygen removal 24 is part of the processing of the second product stream D.

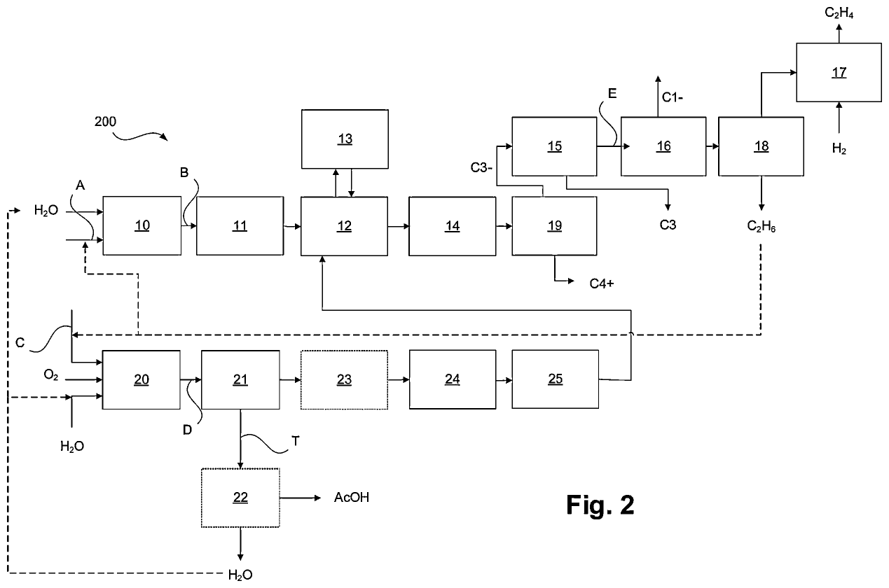

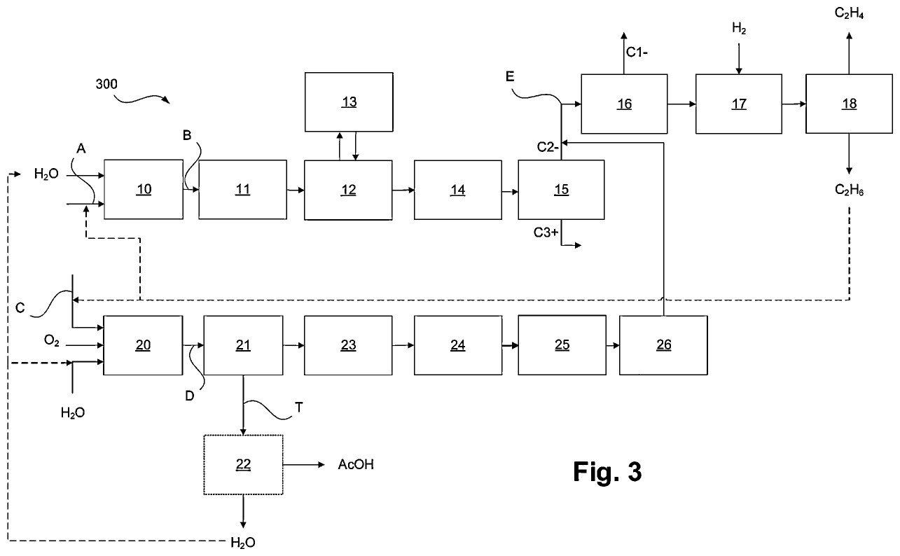

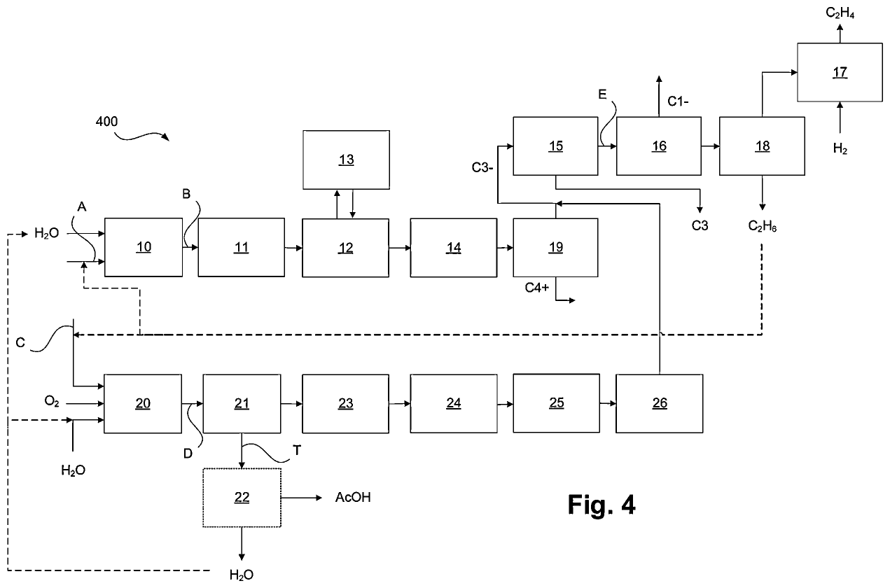

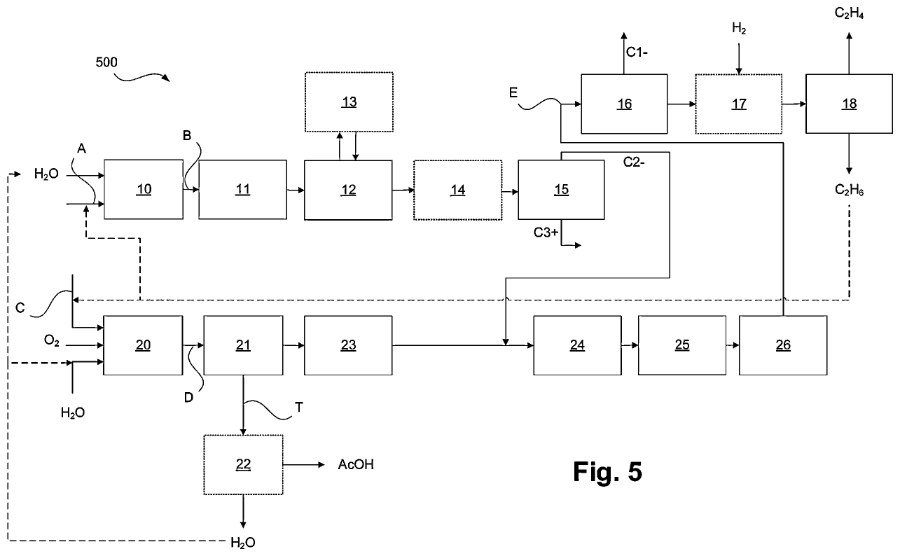

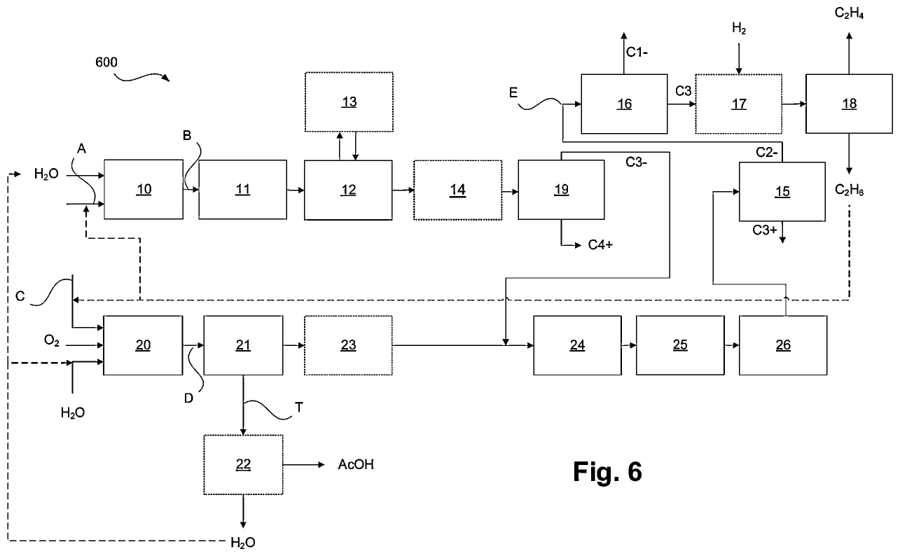

FIGS. 2 to 6 illustrate methods according to further comparative examples not according to the invention (FIGS. 2, 3 and 4) as well as embodiments of the invention (FIGS. 5 and 6) and are each designated overall by 200 to 600. In the following, in particular the differences compared to the embodiment 100 illustrated in FIG. 1 are explained. In each case, optional components or method steps are shown in the form of dashed blocks and are not mentioned as optional in all cases below. In this case, the embodiments according to FIGS. 1 and 2 correspond in particular to “variant 1,” previously mentioned multiple times, the embodiments according to FIGS. 3 and 4 in particular to “variant 2,” previously mentioned multiple times, and the embodiments according to FIGS. 5 and 6 in particular to “variant 3,” previously mentioned multiple times.

In the embodiment 200 illustrated in FIG. 2, the merging is basically carried out in the same way as explained for embodiment 100 according to FIG. 1; a significant difference here is the subsequent processing, as explained in more detail below.

In the embodiment 300 illustrated in FIG. 3, a separate drying 26 is provided in the processing of the second product stream D, i.e., the product stream of the oxidative dehydrogenation 20, which follows the removal 25 of carbon dioxide. A gas mixture obtained here is combined with the light fraction C2− from deethanization 16. Here, too, the demethanization feed stream E is thus formed using a portion of the first product stream B and the second product stream D in each case, and the oxygen removal 24 is part of the processing of the second product stream D.

According to the embodiments 200, 400 and 600, which are illustrated in FIGS. 2, 4 and 6, a depropanization 19 is provided in each case upstream of the deethanization 15, which depropanization is carried out to obtain a heavier fraction C4+ comprising hydrocarbons having four carbon atoms and heavier hydrocarbons, and a lighter fraction C3− comprising hydrocarbons having two and three carbon atoms, methane and optionally other low-boiling components. Accordingly, the heavy fraction C3 from the subsequent deethanization 15 then also contains essentially only hydrocarbons having three carbon atoms from steam cracking 10.

According to the embodiments 200 and 400 illustrated in FIGS. 2 and 4, the tail-end hydrogenation 17 is arranged downstream of the separation 18 from one another of hydrocarbons having two carbon atoms. It is in particular optional in the embodiments illustrated in FIGS. 5 and 6, and is therefore illustrated in the form of dashed blocks.

In the embodiment 400 illustrated in FIG. 4, in the processing of the second product stream D, i.e., the product stream of the oxidative dehydrogenation 20, as in the embodiment 300 according to FIG. 3, a separate drying 26 is provided, which follows the removal 25 of carbon dioxide. A gas mixture obtained here is combined with the light fraction C3− from the depropanization 19. Here too, the demethanization feed stream E is formed using a portion of the first product stream B and of the second product stream D in each case, and the oxygen removal 24 is part of the processing of the second product stream D from the oxidative dehydrogenation.

In the embodiment 500 illustrated in FIG. 5, in contrast to the previously explained embodiments, the light fraction C2− from the deethanization, to which only components of the first product stream B are fed here, is subjected to the oxygen removal 24. Components of the second product stream D, i.e., the product stream of the oxidative dehydrogenation 20, are also fed to this oxygen removal 24. The carbon dioxide removal 25 and the drying 26 follow. A gas mixture taken from the drying 26 is fed to the demethanization 16 in the form of the demethanization feed stream E. Here, too, the demethanization feed stream E is thus formed using a portion of the first product stream B and of the second product stream D in each case. The oxygen removal 24 is part of the processing of the first product stream B and the second product stream D or combined portions thereof.

In the embodiment 600 illustrated in FIG. 6, in contrast to the previously explained embodiments, the light fraction C3− from the depropanization, to which only components of the first product stream B are fed here, is subjected to the oxygen removal 24. Components of the second product stream D, i.e., the product stream of the oxidative dehydrogenation 20, are also fed to this oxygen removal 24. The carbon dioxide removal 25 and the drying 26 follow. A gas mixture taken from the drying 26 is fed to the demethanization 16 in the form of the demethanization feed stream E. Here, too, the demethanization feed stream E is thus formed using a portion of the first product stream B and of the second product stream D in each case. The oxygen removal 24 is part of the processing of the first product stream B and the second product stream D or combined portions thereof.

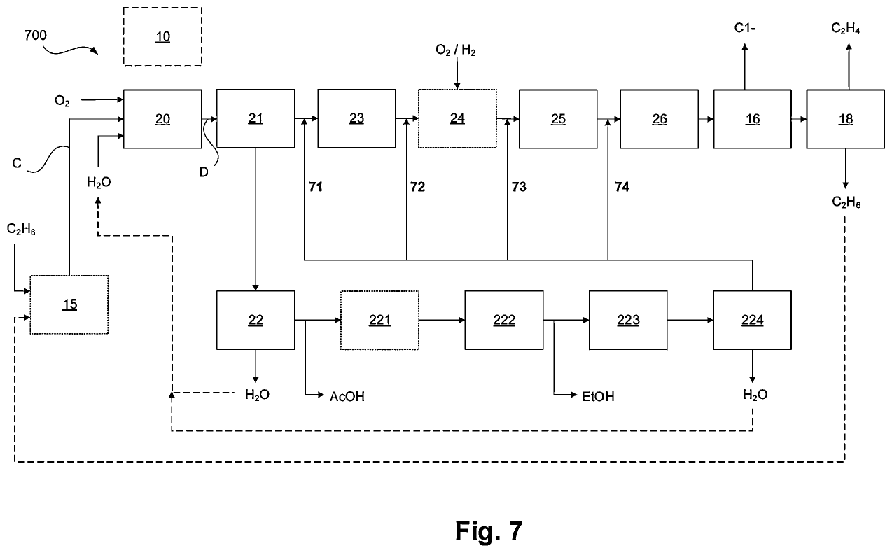

In the context of integration with a steam cracker, where ethylene is a main product, maximizing the ethylene yield may also be desirable. In this case, it is important in particular to avoid acetic acid as a joint product wherever possible. A suitable conversion of acetic acid to ethylene offers a possibility for this, which is described below, by way of example, with reference to FIG. 7. For reasons of generality of the illustration, in this case steam cracking 10 is not illustrated or is shown only as an unconnected block 10.

FIG. 7 illustrates a method according to a corresponding embodiment which is designated as a whole by 700. The method steps illustrated in FIG. 7 are indicated with identical reference signs as above. A repeated explanation is omitted at this point.

In this case, integration into a combination method can be carried out in any way, in particular as explained above. Embodiments of the invention relate in particular to corresponding combination methods. However, the method steps used here can be used not only in embodiments of the invention, but in all combination methods in which an oxidative dehydrogenation 20 is combined with any other methods, in particular, but not limited to, steam cracking 10, and in which corresponding product streams, portions, fractions and the like are combined in any manner or at any positions and thus in particular certain method steps or components are used jointly. In particular, even if demethanization is used in a processing of a product stream from steam cracking 10, hydrogen contained in a top stream from the demethanization can be used in the hydrogenation, in particular after a suitable separation.

As illustrated in FIG. 7, the oxidative dehydrogenation 20, in particular of ethane, is operated here with a downstream condensate separation 21 and a condensate treatment 22. In the ethylene or olefin path, in particular the (pre-)compression 23, in embodiments of the invention an oxygen removal 24, which can be designed in any desired way, a carbon dioxide removal 25, and a drying 26 follow. Demethanization 16 is also provided here. This can be coupled in particular in the manner illustrated above with steam cracking 10, i.e., at a suitable point upstream thereof a product stream B from steam cracking 10 or fractions, portions, etc. can be fed in. A separation of hydrocarbons having two carbon atoms is also designated here by 18. Ethane from this separation can in particular be fed to an optional deethanization 15, where an ethane stream is formed which can be fed at least in part as the (first) feed stream A to the steam cracking, but in particular as the (second) feed stream C to the oxidative dehydrogenation 20.

In particular, a water stream H₂O formed in the condensate treatment can be recycled to the oxidative dehydrogenation 20, like any other water stream formed downstream thereof. Acetic acid (AcOH) can be discharged, but in the embodiment illustrated here at least a portion of it is fed to an optional compression 221 and then fed into an acetic acid hydrogenation 222 in which ethanol (EtOH) is formed. The ethanol can be discharged, but in the embodiment illustrated here at least a portion of it is fed to an ethanol dehydration 223, in which water and ethylene are formed from ethanol. After phase separation, an ethylene stream can be fed into the ethylene path at positions 71 to 74 as required, positions 71 and 72 representing embodiments according to the invention.

In the following, advantages and embodiments of the invention are explained again with explicit reference to the prior art.

The oxidative dehydrogenation of alkanes, in particular of ethane, may be advantageous over more established methods for producing olefins, such as steam cracking or catalytic dehydrogenation. For instance, due to the exothermic nature of the reactions involved and the practically irreversible formation of water, there is no thermodynamic equilibrium limitation. The oxidative dehydrogenation can be carried out at comparatively low reaction temperatures. In principle, no regeneration of the catalysts used is required, since the presence of oxygen enables or causes regeneration in situ. Finally, in contrast to steam cracking, lower amounts of valueless by-products, such as coke, are formed. In embodiments of the invention, oxidative dehydrogenation, in particular of methane, is particularly advantageous because it enables the advantageous use of ethane, even if steam cracking is designed to use (purely or predominantly) liquid feedstocks.

For further details regarding the oxidative dehydrogenation, reference may be made to the specialist literature, for example: Ivars, F. and López Nieto, J. M., Light Alkanes Oxidation: Targets Reached and Current Challenges, in: Duprez, D. and Cavani, F., Handbook of Advanced Methods and Processes in Oxidation Catalysis: From Laboratory to Industry, London 2014: Imperial College Press, pages 767-834, or Gärtner, C. A. et al., Oxidative Dehydrogenation of Ethane: Common Principles and Mechanistic Aspects, ChemCatChem, Vol. 5, no. 12, 2013, pages 3196 to 3247, and X. Li, E. Iglesia, Kinetics and Mechanism of Ethane Oxidation to Acetic Acid on Catalysts Based on Mo-V-Nb Oxides, J. Phys. Chem. C, 2008, 125, 15001-15008.

Particularly relevant aspects of oxidative dehydrogenation in embodiments of the invention and further technical background of corresponding embodiments are explained in more detail below.

The product gas of the oxidative dehydrogenation of ethane after a condensate separation contains significant amounts of acetylene, for example up to 500 vol.ppm, 400 vol.ppm, or 300 vol.ppm and more than 10 vol.ppm, 50 vol.ppm, or 100 vol.ppm, of carbon monoxide, for example up to 5 vol. %, 4 vol. %, or 3 vol. % and more than 0.5 vol. %, or 1 vol. %, of carbon dioxide, for example up to 4 vol. %, 3 vol. %, or 2 vol. % and more than 0.5 vol. % or 1 vol. % and of oxygen, for example up to 2 mol. %, 1.5 mol. %, 1 mol. %, or 0.5 mol. % and more than 500 vol.ppm, 1000 vol.ppm, or 2500 vol.ppm. Apart from acetylene, other mono-and polyunsaturated hydrocarbons (higher olefins, dienes and higher acetylenes) are only present in trace amounts in the product gas.

Options for removing oxygen, previously also referred to as “oxygen removal,” are known in principle and are disclosed, for example, in WO 2020/187572 A1 or in WO 2018/153831 A1. In the case of appropriate oxygen removal, as mentioned above, in particular acetylene can also be removed, as explained below. In this case, residual oxygen contents of less than 1000 vol.ppm, 500 vol.ppm, 100 vol.ppm, 10 vol.ppm, or 1 vol.ppm and acetylene contents of less than 5 vol.ppm, 2 vol.ppm, 0.5 vol.ppm, 0.3 vol.ppm, or 0.1 vol.ppm can typically be achieved. In embodiments of the invention, complete removal of oxygen and acetylene is not necessary.

As described for example in WO 2018/153831 A1, oxygen, carbon monoxide and optionally acetylene can be removed from the product stream of an oxidative dehydrogenation of ethane. In this case, the use of an oxidation catalyst is generally disclosed, which catalyst may contain as components in particular the metals niobium, copper, zinc, palladium, silver, platinum, gold, iron, manganese, cerium, tin, rubidium, and chromium. In this case, copper-and/or platinum-based systems, but in particular copper-based systems, are preferably used. Embodiments of the invention can be carried out using any acetylene and oxygen removal method known from the prior art and the technical literature.

With regard to the removal of oxygen (relevant here without taking other components into account), other approaches are also known. In principle, this can already take place directly at the reactor outlet of the oxidative dehydrogenation of ethane, as disclosed, for example, in WO 2010/115108 A1 or U.S. Pat. No. 8,519,210 B2 as well as WO 2019/175731 A1 or WO 2019/175732 A1. Removal after acetic acid separation and before (e.g., according to WO 2018/153831 A1) or after (e.g., according to WO 2018/153831 A1 or WO 2020/187572 A1) compression is also possible. A corresponding removal directly at the reactor outlet falls under the term “raw gas treatment” explained above.

It is also known to feed hydrogen into such a raw gas treatment of the product gas from an oxidative dehydrogenation of ethane. According to the prior art, only the process gas stream of an oxidative dehydrogenation of ethane is subjected to such a raw gas treatment (in contrast in particular to the repeatedly mentioned “variant 3”). In this case, in particular catalysts containing copper oxide or catalysts containing at least one of the elements copper, silver, gold, manganese, zinc, nickel, platinum, palladium, rhodium, iridium and/or ruthenium can be used in the raw gas treatment.

As mentioned, in the case of corresponding oxygen removal, acetylene and carbon monoxide are usually reduced/removed at the same time. In embodiments of the invention, however, the removal of acetylene takes a back seat (for “variant 1” and “variant 2,” to a limited extent for “variant 3”). However, the conversion of carbon monoxide and the formation of carbon dioxide are relevant for process concepts according to corresponding embodiments of the invention (resulting downstream carbon dioxide removal). However, in particular for acetylene, specification-compliant removal of acetylene can usually be achieved (important for variant 3 without further C2 hydrogenation).

During oxidative dehydrogenation, in particular when using the catalyst systems mentioned, acetic acid is formed as a joint product (typical ratio 3 to 12 mol/mol ethylene to acetic acid), but the demand for acetic acid is often limited to specific downstream products or methods (e.g., the production of vinyl acetate monomer, VAM). Acetic acid can be separated as a component of the condensate phase separated downstream of the oxidative dehydrogenation (the typical acetic acid content in the condensate is 1 to 30 wt. %, 3 to 25 wt. % or 5 to 20 wt. %) and provided as a separate valuable product by suitable processing, as explained.

On the other hand, in contrast to the steam cracker, the oxidative dehydrogenation of ethane additionally produces only ethylene and acetic acid as products-apart from the major by-products of carbon monoxide and carbon dioxide. This also eliminates the requirement for suitable utilization of other product fractions from the oxidative dehydrogenation and the corresponding equipment in the fragmentation part. In embodiments of the invention, the use of corresponding products is particularly advantageously possible.

The technology used in steam cracking is also extensively regulated. What is typically important here in particular is strict compliance with the product specification of ethylene in view of downstream methods (e.g., polyethylene production, ethylene oxide production, etc.). Here, in addition to others, very strict limit values must be observed, in particular for carbon monoxide, carbon dioxide, oxygen and acetylene, which are achieved through appropriate cleaning and separation steps. Particularly relevant process steps are listed below and explained in more detail.

In embodiments of the invention, certain components of the product mixture or raw gas stream in steam cracking and aspects of its further treatment are particularly relevant. In particular, a corresponding product mixture contains a high proportion of polyunsaturated hydrocarbons (dienes and acetylenes) and has a low carbon monoxide and carbon dioxide content. In particular, the carbon monoxide content in the raw gas stream, i.e., essentially immediately downstream of a corresponding reactor or the subsequent quenching, is less than 0.50 vol. %, 0.20 vol. %, or 0.10 vol. %. Typical contents of carbon dioxide in the raw gas stream (before caustic scrubbing) are in particular less than 0.05 vol. %, less than 0.02 vol. %, or less than 0.01 vol. %. The oxygen content should be extremely low or should contain substantially no oxygen.

The oxygen content is in particular less than 10 vol.ppm, in particular less than 1 vol.ppm, in the ethylene product due to strict product specifications of ethylene in view of downstream methods (e.g., production of polyethylene, ethylene oxide, etc.). However, it is also important to limit the oxygen content in the raw gas stream of the steam cracker. This results in particular from the interaction with polyunsaturated hydrocarbons (fouling, especially during compression and also in the bottom of columns).

In principle, different feedstocks can be considered for steam cracking. These comprise light feedstocks such as ethane, propane and what are known as liquefied petroleum gas (LPG), in particular in what are known as gas crackers, but also heavier feedstocks such as naphtha or what is known as atmospheric gasoil (AGO) etc., in particular in what are known as liquid crackers. The crackers are optimized according to the feedstock and differ in part in their design (e.g., raw gas hydrogenation for gas crackers and front-end hydrogenation for liquid crackers). Steam cracking produces a particularly broad range of products (ethylene, propylene, but, in particular in liquid crackers also aromatics, higher hydrocarbons, and heavier fractions). Recycling of higher hydrocarbons, in particular paraffins, into the cracking furnaces is possible. In embodiments of the invention, ethane recycling is particularly relevant. Aspects of the treatment of a raw gas from steam cracking include the removal of carbon monoxide by demethanization (see below) and/or the removal of carbon dioxide, typically by caustic scrubbing (see below).

Selective hydrogenation of hydrocarbons having two carbon atoms serves to reduce the acetylene content in accordance with specifications. In this case, acetylene is selectively hydrogenated to ethylene using suitable catalysts. Selective hydrogenation used to be carried out predominantly using nickel-based catalysts, but nowadays mainly palladium-based catalysts with suitable dopants such as silver, gold, cerium, etc. are used. There is considerable sensitivity to carbon monoxide. Carbon monoxide serves as a moderator and the carbon monoxide content in the hydrogenation is typically in the range of 50 to 2500 vol.ppm, 100 to 1500 vol.ppm or 150 to 1000 vol.ppm. These carbon monoxide contents are significantly lower than the above-mentioned carbon monoxide contents of a process gas from an oxidative dehydrogenation of ethane and also lower than in cases where raw gas treatment is used.

In this case, high carbon monoxide contents in selective hydrogenation can be compensated by a higher temperature, but this may lead to ethylene loss. In this case, the maximum temperature is limited on the one hand by the design temperature of the reactor, in particular in existing systems, but also by the operating window of the catalyst. If the above temperature range is exceeded, selective hydrogenation can no longer be carried out or the use of specially adapted catalysts is required.

In principle, selective hydrogenation is also not insensitive to the presence of oxygen, since water can be formed in the presence of oxygen, which in turn promotes the formation of other by-products such as carboxylic acids and green oil. In embodiments of the invention, corresponding problems are eliminated in a particularly advantageous manner.

Various variants are known as positions for selective hydrogenation:
(1) A crude gas hydrogenation is positioned immediately after compression, caustic scrubbing and drying. This is preferably used in gas crackers, i.e., steam crackers operated with gaseous feedstocks. In this case, higher polyunsaturated hydrocarbons are also converted, at least in part (especially butadiene, methylacetylene and propadiene). Typical carbon monoxide contents are in the lower part of the above range, i.e., in particular in the range of 50 to 1000 vol.ppm or 50 to 500 vol.ppm.
(2) What is known as front-end hydrogenation is positioned after a deethanization. This is therefore a pure acetylene hydrogenation. In both cases, in addition to ethane, corresponding amounts of methane, hydrogen and carbon monoxide are furthermore contained in the feed stream of the selective hydrogenation. Due to the method, typical carbon monoxide contents are higher here than in raw gas hydrogenation, i.e., in particular in the range of 100 to 2500 vol.ppm or 100 to 1500 vol.ppm. Another known variant is the arrangement after depropanization; in this case, the feed stream of the hydrogenation also includes hydrocarbons having three carbon atoms. Similarly to the hydrogenation of raw gas, the corresponding higher polyunsaturated hydrocarbons are then converted at least in part.
(3) In principle, what is known as tail-end hydrogenation is also known, in which at least one stream containing methane and other light components is separated before hydrogenation and a stoichiometric hydrogen dosage is correspondingly required. This variant is particularly relevant in embodiments of the invention in which at least a light fraction C1− is still separated in the demethanization prior to a corresponding tail-end hydrogenation, and a stoichiometric hydrogen addition is accordingly required. Together with this light fraction, lighter molecules and thus in particular carbon monoxide and oxygen are therefore removed from the process stream, so that they have no effect on the tail-end hydrogenation. Precious metal catalysts, which have already been mentioned and can in particular be palladium-based, are also used in tail-end hydrogenation.

In addition to the requirement for high product purity of the ethylene product, cryogenic system parts also require quantitative separation of carbon dioxide in order to avoid freezing of carbon dioxide and thus blockages.

Owing to its strong interaction with suitable solvents or washing liquids, carbon dioxide can likewise be removed comparatively easily from the product mixture, in particular in contents as occur in the case of oxidative dehydrogenation of ethane, it being possible to use known methods for removing carbon dioxide, in particular corresponding scrubbing (for example amine scrubbing). The laden scrubbing liquid is then regenerated in a separate column, and very pure carbon dioxide is released by desorption.

Should subsequent steps require the absence of, or only a very low residual concentration of, carbon dioxide (for example due to catalyst inhibition or what is known as catalyst poisoning), the residual carbon dioxide content after amine scrubbing can be further reduced by an optional caustic scrubbing as fine cleaning, as required.

With certain exceptions, the corresponding scrubbing liquids can also react with oxygen, as a result of which disadvantageous aging or damage to the scrubbing agents, which require a continuous purge and makeup stream or lead to an undesired shortening of the service life of these scrubbing liquids, can occur over time. Therefore, from this aspect of the invention too, the removal of oxygen upstream of a corresponding scrubbing is advantageous.

The raw gas stream from a steam cracker typically contains significantly lower carbon dioxide contents than in oxidative dehydrogenation. These are usually removed using a lye wash. A carbon dioxide content several orders of magnitude higher, as in the oxidative dehydrogenation of ethane, would lead to excessive caustic consumption, as previously stated. In embodiments of the invention, as mentioned, in particular a separate separation of carbon dioxide is therefore carried out by means of amine and optionally alkali scrubbing downstream of the oxidative dehydrogenation.

The removal of water is carried out according to the prior art using regenerative dryers based on molecular sieves and, in addition to achieving the product specification, is also mandatory in view of subsequent cryogenic process steps in order to avoid blockages due to the deposition of ice and hydrates.

Typically, as part of a suitable method control, both in the oxidative dehydrogenation of ethane and in the fragmentation portion of a steam cracker, methane contained in the product stream (e.g., in particular from the ethane feed stream of the oxidative dehydrogenation) must also be removed. This usually involves demethanization, which requires appropriate cryogenic conditions. In this case, demethanization simultaneously removes carbon monoxide and hydrogen from the corresponding stream. A fraction called C1− (pronounced “C1minus”) is thus formed, which contains methane, hydrogen and/or carbon monoxide as essential components. Traces of oxygen still contained in the inlet stream of the demethanization also enter this fraction. Therefore, it is usually sought to limit the inlet oxygen content in the inlet stream for demethanization and thus avoid the possible formation of an explosive atmosphere at the head. A corresponding design of the demethanizer is possible, as described for example in WO 2018/082945 A1, but this means correspondingly significantly increased equipment expenditure. EP 3 456 703 A1 describes a demethanization in the fragmentation portion of a system for the oxidative dehydrogenation of ethane, which is combined with a pressure swing adsorption in the overhead stream. In order to minimize product losses, it is further sought to minimize the ethylene content in the top gas of the demethanizer.

Ultimately, unreacted ethane has to be separated from ethylene, which takes place by means of a C2 splitter, mentioned multiple times, which is likewise operated under cryogenic conditions. This must therefore be constructed and operated in such a way that ethane in the ethylene is practically quantitatively removed (required). Finally, unreacted ethane must be separated from ethylene, which is done by means of a C2 splitter, which is also operated under cryogenic conditions. This must therefore be constructed and operated in such a way that ethane in the ethylene is practically quantitatively removed (required purity of the ethylene product is usually more than 99.9%) and at the same time an ethane stream that is returned for oxidative dehydrogenation contains as little as possible or no ethylene.

Approaches for integration of steam crackers and oxidative dehydrogenation of ethane are basically known, as already mentioned. What all of the above-mentioned documents have in common is that they do not address the well-known formation of acetic acid as a joint product of oxidative dehydrogenation, and in each case only separate an aqueous condensate phase. Embodiments of the invention, in contrast, in particular also take this aspect into consideration.

WO 2018/024650 A1 focuses in particular on the integration of a steam cracker using ethane with an oxidative dehydrogenation of ethane. The feeding of a process gas from an oxidative dehydrogenation of ethane into the fragmentation portion of a steam cracker is in principle described and claimed. However, no solutions are disclosed which address and solve the problem of the significantly different oxygen, carbon monoxide and acetylene contents in the process gas from an oxidative dehydrogenation of ethane and in corresponding streams in the fragmentation portion of a steam cracker. A tail-end hydrogenation and a merging of product streams upstream of this tail-end hydrogenation is described, such that the hydrocarbons having two carbon atoms from steam cracking and oxidative dehydrogenation of ethane are thus subjected to a joint hydrogenation. This is in direct contrast to the approach of embodiments of the invention. Accordingly, no raw gas treatment of the process stream of an oxidative dehydrogenation of ethane is shown or claimed.

WO 2014/134703 A1 discloses oxygen removal immediately downstream of a reactor for the oxidative dehydrogenation of ethane (known as “afterburner”), typical residual oxygen contents of less than 1000 vol.ppm being mentioned. In a general manner, integration approaches of oxidative dehydrogenation of ethane and steam cracking are claimed, which optionally include the process units of C2 splitter and/or acetylene hydrogenation. In this case, the integration can take place both explicitly upstream and explicitly downstream of the acetylene hydrogenation. However, no solutions are presented here for the problem of the significantly different carbon monoxide and acetylene contents in the process gas from an oxidative dehydrogenation of ethane and in corresponding streams in the fragmentation portion of a steam cracker. The advantageous positioning of a demethanizer and integration in the overall method is also not discussed in more detail in the document.

CN 103086821 B concerns the integration of a naphtha cracker with an oxidative dehydrogenation of ethane. There are basic explanations for the removal of oxygen, carbon monoxide and carbon dioxide. However, the document does not disclose a solution for the removal of acetylene. Rather, in contrast to the previous statements, this document teaches that the oxidative dehydrogenation of ethane should produce precisely no joint products such as acetic acid and acetylene.

Embodiments of the invention meet the conflicting requirements outlined above and enable optimized integration of an oxidative dehydrogenation of ethane and a steam cracker.

Specifically, a number of aspects prevent the feeding of a product gas from the oxidative dehydrogenation of ethane into the fragmentation portion of a steam cracker. For example, a high carbon monoxide content in the process gas of the oxidative dehydrogenation of ethane is limiting for the hydrogenation of hydrocarbons having two carbon atoms; here, initially only what is known as tail-end hydrogenation can be used or only special catalysts that tolerate high carbon monoxide content can be used. A carbon dioxide content in the process gas that is too high or the carbon monoxide content argues against removal by means of typical caustic scrubbing. The oxygen content in the process gas of oxidative dehydrogenation can lead to fouling effects, a possible enrichment of oxygen in the mentioned C1− fraction, and therefore to a safety hazard. In this case, even after a separate raw gas treatment in the process gas of the oxidative dehydrogenation, the oxygen content still remains critical due to polyunsaturated hydrocarbons from the cracker, or there is a very strict purity requirement (less than 10 vol.ppm, in particular less than 1 vol.ppm oxygen) for such a raw gas treatment, which requires corresponding effort and can lead to ethylene losses.

Embodiments of the invention solve these problems in the manner explained.

If, in the comparative examples not according to the invention that are explained above, oxygen is already largely removed in the process gas of the oxidative dehydrogenation of ethane by means of a raw gas treatment in the previously explained non-inventive comparative examples, it is possible to feed it in upstream of a caustic wash (“variant 1”). This caustic wash can then also be used in particular to remove carbon dioxide residues in the process gas from the oxidative dehydrogenation of ethane (fine cleaning). If only a partial reduction of the oxygen content in the process gas of the oxidative dehydrogenation of ethane is achieved, the feed can be carried out in particular downstream of a deethanization or depropanization (“variant 2”) in order to avoid fouling reactions involving oxygen and dienes.

In embodiments of the invention, it is provided that the oxygen removal takes place only after the aforementioned combining (“variant 3”). In this case, in particular the proposed combining downstream of at least one deethanization or depropanization of the product stream of steam cracking followed by a raw gas treatment of the combined process stream is particularly advantageous.

In particular in “variant 2” and “variant 3,” remaining oxygen can be removed via a subsequent demethanizer. Impurities caused by acetylene can be reduced or removed at least in part via the raw gas treatment; a final removal in accordance with the specifications is possible by means of a selective hydrogenation mentioned above, which, according to embodiments of the invention, can be carried out in particular as a tail-end hydrogenation (i.e., after separation of carbon monoxide in the demethanization).

In the following, the comparative examples not according to the invention, and embodiments of the present invention, in particular according to “variant 1,” “variant 2,” and “variant 3,” are summarized again.

A pretreatment of the second product stream of the oxidative dehydrogenation of ethane (only “variant 1” and “variant 2,” the “variant 3” according to embodiments of the invention advantageously does not require any separate pretreatment of the second product stream) comprises in particular an oxygen reduction by a raw gas treatment (in this case a reduction of acetylene and carbon monoxide takes place at the same time) and in particular a pre-compression (optional for “variant 1”) upstream and/or downstream of the raw gas treatment. Furthermore, in a corresponding pretreatment of the second product stream of the oxidative dehydrogenation of ethane, in particular a separate carbon dioxide removal is provided (in particular by means of amine scrubbing; here carbon dioxide is advantageously obtained as a separate pure product for possible further use and/or carbon capture & storage). In “variant 1,” in particular, only carbon dioxide is removed by means of amine scrubbing and then a joint fine cleaning takes place after combining with the first product stream from the steam cracker. For “variant 2”, the carbon dioxide removal from the second product stream of the oxidative dehydrogenation of ethane can in particular also be carried out in two stages (with regenerative or amine scrubbing and caustic scrubbing) in order to achieve similarly low carbon dioxide contents to those in the latter, downstream of the caustic scrubbing, before combining with the first product stream from the steam cracker and as required for feeding into cryogenic system parts or for compliance with the ethylene product specification (usually values of less than 1 mol-ppm or even lower). Furthermore, in a corresponding pretreatment of the second product stream of the oxidative dehydrogenation of ethane (only in “variant 2”), in particular drying is provided.

As mentioned, combining of the corresponding product streams or portions thereof according to comparative examples not according to the invention takes place upstream of a demethanization, specifically according to “variant 1” upstream of a carbon dioxide removal (in particular caustic scrubbing, preferably used here as a common fine cleaning), as mentioned multiple times in particular a corresponding pre-cleaning of the second product mixture of the oxidative dehydrogenation of ethane taking place beforehand. In particular, the combining step is optionally carried out upstream of a compression or individual compression stages which are connected upstream of a corresponding carbon dioxide removal, and the joint carbon dioxide removal is followed in particular by drying.

According to “variant 2” not according to the invention, and “variant 3” according to embodiments of the invention, corresponding combining takes place in particular downstream of a deethanization or depropanization of the first product mixture from the steam cracking, since dienes (in particular butadiene) are no longer present here. Depending on the design, drying of the common stream downstream of the aforementioned separation is possible here (in particular in the case of infeed downstream of a depropanization).

In addition (in particular only for “variant 3” according to embodiments of the invention) a treatment of the mixed stream after the combining, by means of a raw gas treatment in the sense explained above, is provided, which at least reduces the oxygen content. In particular, in this case acetylene and carbon monoxide are also converted at least in part, and acetylene is advantageously removed according to specifications. Furthermore, carbon dioxide removal is provided in particular here, in order to avoid blockages due to carbon dioxide freezing out in the cryogenic part and to comply with the maximum carbon dioxide content in the ethylene product specification.

Necessary adjustments of the temperature levels are not explicitly taken into account here and in FIGS. 1 and 2 explained above, and are carried out in a manner known to a person skilled in the art (e.g., heat exchanger).

In one embodiment of the invention, particularly preferably at least portions of the combined product gas stream are cooled at individual points in the process to temperatures of less than −120° C., −135° C., or −150° C. The achievement of particularly low temperatures promotes the minimization of ethylene losses in the light fraction C1−, which, as mentioned, is separated from the combined product gas stream in the demethanizer.

This results in an additional synergy advantage of the integration, since in a stand-alone, non-integrated system for oxidative dehydrogenation of ethane proportionally only a small C1minus fraction is available. This is not suitable for the generation of usable peak cold, and temperatures below −110° C. cannot be achieved in a stand-alone oxidative dehydration system without considerable additional effort. This situation improves significantly if the product gas stream from the oxidative dehydrogenation of ethane is combined with that from the cracker and fed to a common demethanizer. Thus, ethylene losses can be reduced at least in part for the fraction of oxidative dehydrogenation of ethane by the availability of peak cold in the above-mentioned temperature range.

An increase in the ethylene yield is possible by converting acetic acid from a condensate separated downstream of the oxidative dehydrogenation to ethylene. As mentioned, corresponding method steps can be used not only in embodiments of the invention, but in all combination methods in which the oxidative dehydrogenation is combined with any other methods, in particular, but not limited to, steam cracking, and in which corresponding product streams, portions, fractions and the like are combined in any manner or at any positions and thus in particular certain method steps or components are used jointly.

As mentioned, during oxidative dehydrogenation, in particular when using MoVNbOₓ-based and in particular MoVNbTeOₓ catalysts under industrially relevant reaction conditions, significant amounts of the respective carboxylic acids of the paraffins used are formed as by-products. For economic system operation, a corresponding coupled production of olefins and of the respective carboxylic acids is generally required when using the catalyst type described. This applies in particular to the production of ethylene by the oxidative dehydrogenation of ethane in which acetic acid is formed at the same time.

In the context of the corresponding embodiment, in particular the mentioned formation of acetic acid as a joint product is relevant. Reference is made to the explanations above. An adjustment of the ratio of ethylene to acetic acid from the oxidative dehydrogenation of ethane is possible in particular by adjusting the water content, in particular the water partial pressure in the process gas stream, but only within certain limits (cf. e.g., WO 2018/115416 A1 or EP 3 519 377 B1). Furthermore, a certain minimum water content is advantageous or necessary to ensure stable catalyst performance (cf. e.g., WO 2018/115418 A1 or EP 3 558 910 B1). The use of oxidative dehydrogenation of ethane is mostly limited to small to medium system capacities due to the coupled production, and requires corresponding utilization of the acetic acid produced. On the other hand, however, as already mentioned, in contrast to steam cracking—apart from the main by-products carbon monoxide and carbon dioxide—only ethylene and acetic acid are produced as products. This also eliminates the requirement for suitable utilization of other product fractions from the oxidative dehydrogenation of ethane and the corresponding equipment in the fragmentation portion.

A system for the oxidative dehydrogenation of ethane typically comprises the process steps already explained multiple times. In this case, the acetic acid formed in the oxidative dehydrogenation of ethane can first be hydrogenated to ethanol and then the ethanol can be dehydrated to ethylene. The ethylene formed can be fed into a corresponding method or a corresponding processing sequence at any position downstream of the oxidative dehydrogenation of ethane.

The hydrogenation of carboxylic acids is known and is described, for example, in the article “Hydrogenation and Dehydrogenation” in Ullmann's Encyclopedia of Industrial Chemistry, 2012 edition. Water is formed as a coproduct of the hydrogenation. The article in question in particular discusses the requirement of increased pressure and increased temperature for the hydrogenation of carboxylic acids. In particular rhenium, ruthenium, copper and chromium are listed as catalyst components.

Corresponding specific embodiments of the hydrogenation can also be found, for example, in EP 0 100 406 B1 using cobalt-containing catalysts at 10 to 350 bar and 210 to 330° C. in the gas phase. Other possible catalyst components include manganese and molybdenum.

WO 2010/014153 A2 is also based on a cobalt catalyst, but in this case other components are used, in particular selected from palladium, platinum, rhodium, rubidium, rhenium, iridium, cerium, copper, tin, molybdenum, tungsten, vanadium, and zinc. In addition to the catalyst composition, WO 2011/056597 A2 also discloses details on the method control of acetic acid hydrogenation and further processing of the ethanol product. WO 2011/097190 A3, for example, also discloses details of the method control with the aim of maximizing ethanol yield. WO 2013/101373 A1 discloses ethyl acetate recycling in acetic acid hydrogenation. In addition to the previously mentioned possible catalyst components, iron, lanthanum, cerium and gold are also mentioned here. In WO 2013/122645 A1, nickel, osmium and caesium are also added.

The hydrogenation of acetic acid in the aqueous phase is described in Y. Zhao et al., Catalysts 2020, 10, 1270 ff. A ruthenium-tin/titanium dioxide-based catalyst is used. In particular at temperatures below 220° C., very high ethanol yields well over 90% are described. The publication contains further references to the hydrogenation of acetic acid in the aqueous phase.

The aforementioned documents therefore cover both the use of pure or highly concentrated acetic acid as a reaction feed, and the use of aqueous solutions of acetic acid. In this case, the catalysts mentioned can be used either as solid material or as supported catalysts, e.g., on materials containing dialuminium trioxide, silicon dioxide, zirconium dioxide, titanium dioxide, carbon, etc.

The dehydration of alcohols such as ethanol at suitable catalysts to produce the corresponding olefins is also known and described, for example, in DE 10 2019 119 540 A1. In particular, the production of ethylene (from ethanol) is common and is gaining importance in connection with the increasing production quantities of (bio)ethanol. For example, reference is made here to the article “Propanols” in Ullmann's Encyclopedia of Industrial Chemistry and Intratec Solutions' “Ethylene Production via Ethanol Dehydration,” Chemical Engineering 120, 2013, 29. The dehydration is very easy to carry out in the presence of mineral acid catalysts at room temperature or above. The reaction itself is endothermic and equilibrium limited. High conversions are favored by low pressures and high temperatures. Technically, a plurality of adiabatic reactors connected in series with appropriate intermediate heating are usually used.

Typically, heterogeneous catalysts based on dialuminium trioxide or silicon dioxide are used in this case. In general, a plurality of types of acid catalysts are suitable, and for example molecular sieves and zeolites can also be used. Typical temperatures range from 200 to 250° C. for the dehydration of ethanol or 300 to 400° C. for the dehydration of 2-propanol or butanol. Owing to the equilibrium limiting, the product stream is typically separated off (separation of the olefin product and also at least in part of the water, for example by distillation) and the stream containing unconverted alcohol is recycled to the reactor inlet. In this way, overall very high selectivities and yields can be achieved.

Corresponding embodiments, in particular for higher alcohols, are described, for example, in WO 2015/181302 A1. EP 2 740 718 A1 relates to the dehydration of ethanol at increased pressure of 25 to 80 bar, while DE 10 2011 102 971 A1, for example, discloses the advantageous pressure increase of the ethanol feed of a dehydration by means of a pump.

All of the techniques explained can be used to solve the problem of purposeful utilization of unwanted acetic acid as a joint product of oxidative dehydrogenation. In addition, the amount of acetic acid can be reduced practically at will, so that only the actual current demand for acetic acid is provided, for example in an integrated system complex.

This eliminates in particular the limitation of coupled production, as the ratio of ethylene to acetic acid can be adjusted only within certain limits by varying the process conditions, in particular the water content in the reaction feed or the residence time and the pressure. Conventional methods for ethylene production (in particular steam cracking with fired furnaces) are associated with corresponding carbon dioxide emissions.

By a clever combination of the method steps of oxidative dehydrogenation of ethane, optional preparation of acetic acid, acetic acid hydrogenation, and optional ethanol dehydration, a needs-based and virtually freely selectable reduction in the amount of acetic acid is achieved. In this case, on the one hand ethanol can be obtained as a valuable additional product, or the ethanol can be further dehydrated to ethylene.

In one embodiment, hydrogen from electrolysis can be used at least in part for the hydrogenation. This enables the use of electricity from renewable sources and avoids additional carbon dioxide emissions. Depending on the requirements, the acetic acid can be concentrated before hydrogenation, but in principle hydrogenation in the aqueous phase is also known and possible. Any pressure increase that may be necessary in this case, in particular for hydrogenation, can be achieved particularly advantageously in this case by using a pump, due to the low equipment and energy expenditure.

The dehydration can also be carried out particularly advantageously at increased pressure, so that no further increase in pressure of the ethylene stream from the dehydration is necessary downstream of the dehydration.

Embodiments have already been explained above with reference to FIG. 7. In this case, different variants 71 to 74 are shown for feeding in an ethylene fraction obtained in the dehydration, namely upstream of a compression or individual compression stages, upstream of a raw gas treatment (optional), upstream of a carbon dioxide removal, and/or upstream of a drying.

The raw gas treatment shown is optional and can be omitted or replaced or supplemented for example by selective hydrogenation at a suitable point in the process. The raw gas treatment can also take place at another point, in particular before compression or before individual compressor stages. In the case of infeed at positions 71, 72 and 73, carbon dioxide removal takes place in each case. According to variant 73, a pressure increase after dehydration is not necessary.

The positions of the demethanizer and splitter can in principle also be swapped, as already explained above.

A separate pressure increase of the ethylene stream after dehydration and phase separation, which is optionally required in variants 72, 73 and 74, is not shown. However, such compression of this stream can be dispensed with and a pressure increase can be used in the use of acetic acid hydrogenation and/or ethanol dehydration. There, the pressure increase can advantageously be carried out in particular in a liquid phase by means of a pump.

In a further embodiment of the invention, a deethanization upstream of the reactor for oxidative dehydrogenation is used, in order to separate heavier components from the reaction feed of the oxidative dehydrogenation. This can be advantageously used to also separate higher components from the dehydration product stream, since these accumulate in the bottoms stream of a C2 splitter.

1. A method for producing one or more hydrocarbons, comprising:
subjecting a first feed stream to steam cracking to obtain a first product stream;
subjecting a second feed stream containing ethane to oxidative dehydrogenation to obtain a second product stream;
subjecting at least a portion of the first product stream to deethanization or depropanization separately from the second product stream to obtain a lighter fraction and a heavier fraction;
forming a demethanization feed stream by combining at least a portion of the lighter fraction and at least a portion of the second product stream; and
subjecting the demethanization feed stream to demethanization at least in part, wherein:
at least partial oxygen removal is carried out during the formation of the demethanization feed stream, the oxygen removal being carried out downstream of the combining step;
at least a portion of the first product stream is fed to the combining step without prior acetylene hydrogenation or with only partial acetylene hydrogenation; and
the oxygen removal comprises an acetylene removal.

2. The method according to claim 1, further comprising:
forming a collective stream by combining at least a portion of the first product stream and at least a portion of the second product stream without prior separation of gaseous hydrocarbons; and
subjecting the collective stream at least in part to carbon dioxide removal;
wherein, the demethanization feed stream is formed using at least a portion of a withdrawal stream taken from the carbon dioxide removal.

3. The method according to claim 2, further comprising:
subjecting the second product stream to a condensate separation and/or a pre-compression upstream of the combining step, and/or;
wherein one or more method steps selected from the group consisting of a carbon dioxide remove, a drying, and a hydrocarbon fractionation is or are carried out downstream of the oxygen removal and upstream of the demethanization.

4. The method according to claim 1, wherein a fraction is formed in the demethanization which predominantly or exclusively contains hydrocarbons having two carbon atoms and which is subjected to a separation of the hydrocarbons having two carbon atoms from one another after or before a selective hydrogenation of acetylene.

5. The method according to claim 1, wherein the oxidative dehydrogenation is carried out using one or more catalysts containing the metals molybdenum, vanadium, and niobium.

6. The method according to claim 1, wherein the carbon dioxide removal downstream of the oxygen removal comprises regenerative scrubbing.

7. The method according to claim 1, wherein the oxygen removal is carried out using one or more catalysts comprising one or more elements selected from the group consisting of copper, silver, gold, manganese, zinc, platinum, palladium, rhodium, iridium, and ruthenium.

8. The method according to claim 1, wherein the oxygen removal is carried out in such a way that no explosive mixture results in a light fraction formed in the demethanization.

9. The method according to claim 1, which comprises a hydrogenation of acetic acid formed in the oxidative dehydrogenation and a dehydration of ethanol formed in particular in the dehydrogenation of the acetic acid.

10. A system for producing one or more hydrocarbons, the system being configured to:
subject a first feed stream to steam cracking to obtain a first product stream and a second feed stream containing ethane to oxidative dehydrogenation to obtain a second product stream;
form a demethanization feed stream using at least a portion of the first product stream and using at least a portion of the second product stream and to subject this to demethanization at least in part;
carry out at least a partial removal of oxygen during the formation of the demethanization feed stream;
subject at least a portion of the first product stream to deethanization or depropanization separately from the second product stream to obtain a lighter fraction and a heavier fraction,
form the demethanization feed stream by combining at least a portion of the lighter fraction and at least a portion of the second product stream; and
carry out the oxygen removal downstream of the combining step,
feed at least a portion of the first product stream to the combining step without prior acetylene hydrogenation or with only partial acetylene hydrogenation; and
carry out the oxygen removal as comprising an acetylene removal.

11. (canceled)

12. (canceled)

13. (canceled)

14. The method according to claim 1, further comprising:
subjecting the second product stream to a condensate separation and/or a pre-compression upstream of the combining step;
wherein one or more method steps selected from the group consisting of a carbon dioxide remove, a drying, and a hydrocarbon fractionation is or are carried out downstream of the oxygen removal and upstream of the demethanization.

15. The method according to claim 1, wherein one or more method steps selected from the group consisting of a carbon dioxide remove, a drying, and a hydrocarbon fractionation is or are carried out downstream of the oxygen removal and upstream of the demethanization.

16. The method according to claim 2, wherein one or more method steps selected from the group consisting of a carbon dioxide remove, a drying, and a hydrocarbon fractionation is or are carried out downstream of the oxygen removal and upstream of the demethanization.

17. The method according to claim 5, wherein the one or more catalysts further contain the metal tellurium.

18. The method according to claim 1, which comprises a dehydration of ethanol formed in particular in the dehydrogenation of the acetic acid.
